(19) United States
(12) Patent Application Publication
(10) Pub. No.: US 2009/0036932 A1
(43) Pub. Date: Feb. 5, 2009

(54) Fixation kit for medical or surgical use

(76) Inventors: Guillaume Francois Rouyer, Massieux (FR); Eric Stephane Fourcault, Ecully (FR); Bertrand Xavier Francois Gauneau, Dardilly (FR)

Correspondence Address: JENKINS, WILSON, TAYLOR & HUNT, P. A., Suite 1200 UNIVERSITY TOWER, 3100 TOWER BLVD.,, DURHAM, NC, 27707, US

(21) Appl. No.: 12/214,800

(22) Filed: Jun. 20, 2008

(30) Foreign Application Priority Data
Jun. 21, 2007 (FR) 0704449

(51) Int. Cl. A61B 17/70; A61B 17/04

(52) U.S. Cl. 606/280; 606/301; 606/286; 606/289

(57) ABSTRACT

The invention relates to a fixation kit (1) comprising an assembly part (2) comprising a housing comprising a first and second fixation member each comprising a head, said kit being characterized in that said housing is designed to cooperate with either of the fixation members and being shaped to create a seat against which the head is intended to rest, the seat and the head being shaped so that the cooperation of the seat and the head of the first fixation member locks the latter, and so that the cooperation of the seat and the head of the second fixation member enables a variation of the orientation thereof.

### RELATED APPLICATIONS

This application claims the benefit of French Patent Application Serial No. FR 07 04449, filed Jun. 21, 2007, the disclosure of which is incorporated herein by reference in its entirety.

1. Technical Field

The present invention relates to the field of devices intended to be fixed to a substrate, with a view for example to consolidating or repairing said substrate, or providing said substrate with an additional functional component consisting of the device.

More specifically, the present invention relates to the medical field, particularly the field of surgical devices, especially bone surgery, that can be used for example in orthopedic surgery, traumatology, or reconstructive surgery.

2. Background

In this way, the present invention relates to a fixation kit for medical or surgical use comprising an assembly part intended to be coupled with a biological substrate and comprising at least one first through housing provided therein, said kit comprising a first and a second fixation member each comprising a head and a shaft intended to be anchored in said substrate.

It is already known how to provide osteosynthesis using a kit comprising, on one hand, an assembly plate provided with slots and, on the other, fixation screws intended to be received in said slots to attach the plate with the bone substrate to be treated.

In order to obtain satisfactory therapeutic efficacy, it is important to ensure firm fixation of the screw with respect to the plate, to prevent mobility between the screw and the plate which would have an adverse effect on the rigidity of the bone assembly produced and could even result in extraction of the screws. To this end, it is conventional to use kits comprising, on one hand, tapered head screws, i.e. wherein the head displays a tapered shape bottom face and, on the other, plates wherein each slot defines a tapered bearing surface for the corresponding screw head.

In this way, each screw head rests, via the tapered bottom face thereof, against a complementary tapered bearing surface, thus producing a surface support suitable for limiting the mobility of the screw head with respect to the plate, once the screw is in place.

However, these conventional kits are not suitable for certain situations requiring positioning of one or more screws according to a specific orientation (angulation) with respect to the plate, said orientation being for example dependent on the specific bone configuration in question and/or the nature and geometry of the lesions to be treated.

In this case, the use of a conventional kit similar to those described above is not suitable, as it does not enable the practitioner to choose the orientation of the screw with respect to the plate. In these conventional kits, the incidence of the screws is in fact fixed and predefined by the orientation of the tapered bearing surface guiding the screw head.

In order to remedy this problem, kits have been proposed wherein the slots of the plate define spherical bearing surfaces, whereas the associated screw heads themselves have a spherical shape. This makes it possible to obtain ball joint type guiding between the screw head and the bearing surface against which the screw head rests. Such spherical guiding makes it possible to vary the orientation of the screw with respect to the plate in a solid angle such that the practitioner is able to choose the angulation of the screw.

However, these orientable screw osteosynthesis kits according to the prior art suffer from a less satisfactory attachment level between the head and the plate than that obtained with conventional kits with tapered bearing surfaces and screws described above.

This relative attachment weakness is naturally due to the very nature of the connection between the spherical screw head and the corresponding spherical bearing surface, which specifically allows a certain angular freedom of the screw.

This attachment weakness between the spherical screw head and the corresponding spherical bearing surface is all the more regrettable as, in a large number of operative situations, the possibility of angular mobility of the screws is only used for a small minority, the other screws not necessarily needing a specific orientation and being generally fixed according to a normal orientation with respect to the plate. In other words, the orientable screw kits according to the prior art offer an angular orientation function available for each slot provided in the plate, while this function is in practice not always necessary, or is only necessary on a few slots (the number and location of which are however not known in advance). Naturally, for slots on which the possibility of angular variation is not required, it would have been preferable to have tapered guiding in that the latter enables improved stability of the attachment between the head and the plate.

However, in many operative situations, the slots whereon an angular orientation possibility is required are not generally known in advance, either in terms of number or location on the plate. Conversely, the slots whereon it will be necessary to provide an excellent attachment of the screw head with the plate are not known in advance either.

This requires surgeons to have several types of kits (with orientable screws and with non-orientable screws), which complicates hospital management and therefore increases the costs associated with this management.

Above all, this results in surgeons making a compromise in terms of fixation, favoring in each individual case one function (for example angular mobility) over another (for example attachment stability), which often does not make it possible to offer the patient an optimal therapeutic effect.

Therefore, the aim of the invention is to remedy the various drawbacks listed above and propose a new fixation kit for medical or surgical use that is simple, economical and wherein the design makes it possible to produce an assembly offering an excellent compromise between rigidity and stability, on one hand, and adaptability to anatomical constraints, on the other.

Another aim of the invention is to propose a new fixation kit for medical or surgical use which makes it possible to obtain a particularly stable assembly and which offers an excellent extraction resistance.

Another aim of the invention is to propose a new fixation kit for medical or surgical use with a particularly simple and reliable design.

Another aim of the invention is to propose a new fixation kit for medical or surgical use making it possible to obtain a removable assembly wherein the stability and extraction resistance are optimized.

The aims assigned to the invention are achieved using a fixation kit for medical or surgical use comprising an assembly part intended to be coupled with a biological substrate and comprising at least one first through housing provided therein, said kit comprising a first and a second fixation member each comprising a head and shaft intended to be anchored in said substrate, said kit being characterized in that said first housing is designed to cooperate equally well with either of said first and second fixation members according to the user's choice, said housing being shaped to create a seat comprising a bearing surface of substantially tapered shape against which the head of the selected fixation member is intended to rest, the seat and the head of each fixation member being shaped with respect to each other so that, on one hand, the cooperation of the seat and the head of the first fixation member substantially locks the latter in a predetermined orientation with respect to the assembly part, the head of the first fixation member comprising a bottom face of substantially tapered shape intended to rest against the bearing surface of substantially tapered shape, and so that, on the other, the cooperation of the seat and the head of the second fixation member enables a variation of the orientation thereof with respect to the assembly part.

Other aims and advantages of the invention will emerge in more detail on reading the description hereinafter, and using the appended figures provided for purely illustrative and non-limitative purposes, wherein:

### BRIEF DESCRIPTION OF THE DRAWINGS

FIG. 1 illustrates, in a general perspective view, an example of a fixation kit according to the invention, said kit being intended to be implanted surgically to perform osteosynthesis.

FIG. 2 is an enlarged sectional view along the line A-A of FIG. 1.

FIG. 3, illustrates, in an enlarged partial sectional view, the cooperation of the first housing of the assembly part with, on one hand, a countersunk head bone screw forming the first fixation member and, on the other, a flat threaded pin forming the first locking member.

FIG. 4 illustrates, in an enlarged partial sectional view, the cooperation of the first housing of the assembly part with, on one hand, a spherical head bone screw forming the second fixation member and, on the other, a convex threaded pin forming the second locking member.

FIG. 5 illustrates, in a perspective view, the first fixation member alone, which in this case consists of a countersunk head bone screw.

FIG. 6 illustrates, in a perspective view, the second fixation member alone, which in this case consists of a spherical head bone screw.

FIG. 7 illustrates, in a perspective view, a flat threaded pin intended to form a first locking member for the screw illustrated in FIG. 5.

FIG. 8 illustrates, in a sectional view, the flat threaded pin in FIG. 7.

FIG. 9 illustrates, in a perspective view, a convex threaded pin forming the second locking member for the screw illustrated in FIG. 6.

FIG. 10 illustrates, in a sectional view, the convex threaded pin in FIG. 9.

### DETAILED DESCRIPTION

The invention relates to a fixation kit 1 for medical or surgical use. The fixation kit 1 according to the invention is preferentially intended to be implanted in the human body, by means of surgery. In this way, the alternative embodiment of the kit 1 according to the invention illustrated in the figures is intended to be used in orthopedic surgery or in traumatology to perform osteosynthesis, with a view for example to reducing a bone fracture.

The kit 1 according to the invention comprises an assembly part 2 intended to be coupled with a biological substrate (not illustrated). In the example illustrated in the figures, the assembly part 2 is a surgical implant, and more specifically an osteosynthesis surgical plate.

In this example, the biological substrate whereon the assembly part 2 is intended to be coupled therefore consists of bone. In this case, the plate forming the assembly part 2 is intended to provide a mechanical connection between the various fragments of the fractured bone, as is known per se.

However, the invention is not limited to an assembly part 2 consisting of an osteosynthesis plate and, for example, it is possible to envisage the assembly part 2 consisting of an arthrodesis plate (to immobilize a joint) or consisting of a cotyloid implant or an implant of any kind, or even ancillary surgical material.

The invention is also not limited to a biological substrate consisting of bone and may relate to other biological substrates, including relatively soft tissues, or at the very least displaying a lower rigidity than that of bones.

Preferentially, the assembly part 2 is all in one piece, i.e. it consists of a single-piece unit. The assembly part 2 may be made of any implantable material having compatible mechanical characteristics with the desired use. In this way, if the assembly part 2 forms, as illustrated in the figures, an osteosynthesis plate, said plate may for example be made of a metallic material such as stainless steel or titanium.

According to the invention, the assembly part 2 comprises at least one first through housing 3 provided therein. In other words, the first housing 3 forms a slot which passes through the assembly part 2 and opens on either side thereof.

In the example illustrated in the figures, the first housing 3 thus consists of an orifice produced on either side of the assembly part 2, throughout the thickness thereof, between its top face 2A and its opposite bottom face 2B. Said bottom face 2B is intended to be positioned facing the biological substrate to be consolidated.

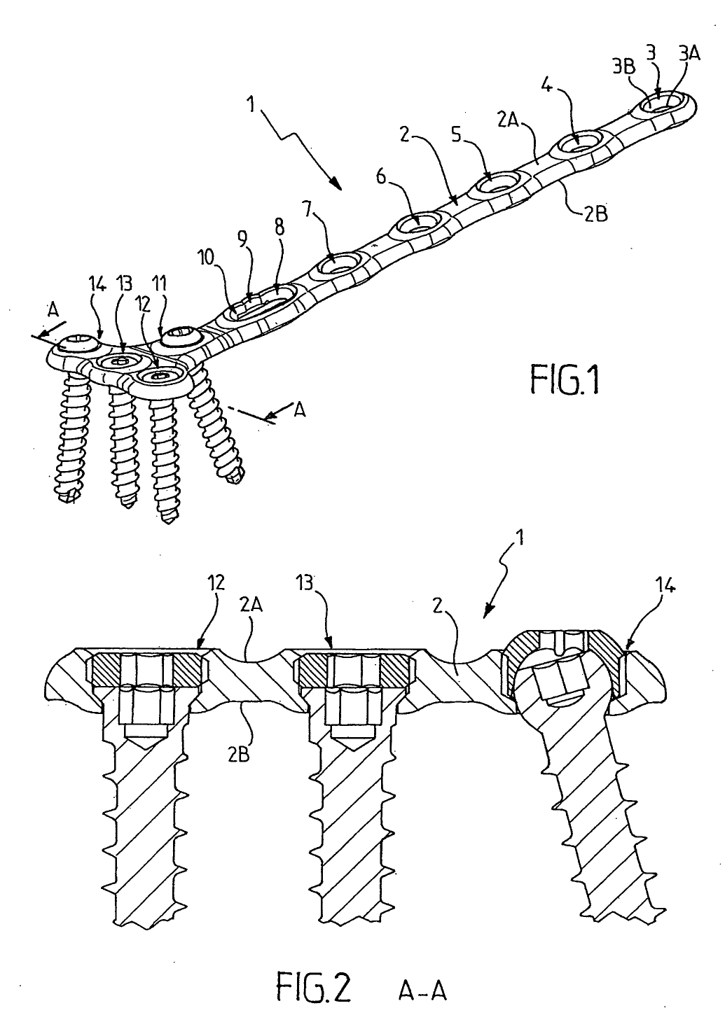

Advantageously and as illustrated in FIG. 1, the assembly part 2 comprises, in addition to the first through housing 3, at least one second through housing 4, of a substantially similar, and preferentially identical, design to that of the first housing 3. In the example illustrated in FIG. 1, the assembly part 2 even comprises a plurality of through housings 3, 4, 5, 6, 7, 11, 12, 13, 14 identical to each other and distributed on the surface of the assembly part 2. In the same example, the assembly part 2 comprises, in addition to the nine identical through housings 3, 4, 5, 6, 7, 11, 12, 13, 14, three housings 8, 9, 10 communicating with each other in the manner described in the French patent application published under the number FR 2 886 535 A1.

It can of course be envisaged for the assembly part 2 to comprise other housings of different designs, without leaving the scope of the invention.

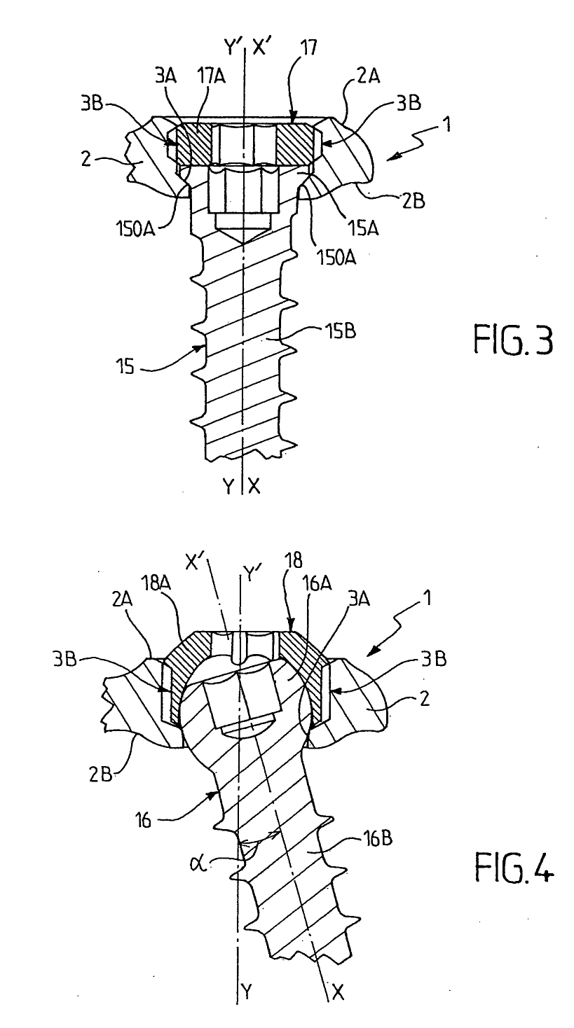

According to the invention, the kit 1 comprises a first and a second fixation member 15, 16, the first housing 3 being designed to cooperate equally well with either of said first and second fixation members 15, 16, according to the user's choice. Preferentially, the first housing 3 is thus designed to cooperate individually with the first and second fixation members 15, 16, which are illustrated in FIGS. 3 and 4, respectively.

The first and second fixation members 15, 16 each comprise, as known per se, a head 15A, 16A and a shaft 15B, 16B extending longitudinally from the head 15A, 16A along a main extension axis X-X′. The shafts 15A, 16A are naturally intended to be anchored in the biological substrate.

Preferentially, said first and second fixation members 15, 16 respectively consist, as illustrated in FIGS. 3 and 4, of a first and second screw, said screws being in this case bone screws. In this case, the top of the head 15A, 16A of each fixation member comprises a clamping imprint, of the hexagonal imprint type, intended to receive a corresponding wrench with a view to tightening the shaft 15A, 16A in the biological substrate.

Naturally, the invention is not limited to the use of fixation members 15, 16 consisting of screws and it is possible to envisage said fixation members 15, 16 consisting of any other alternative means well known to those skilled in the art, for example studs or pins.

According to the invention, the first housing 3 is shaped to produce a seat 3A against which the head 15A, 16A of the selected fixation member 15, 16 is intended to rest, as particularly illustrated in FIGS. 2 to 4.

In other words, in the embodiment illustrated in the figures, the first housing 3 is designed to individually receive the head 15A, 16A of either of the first and second fixation members 15, 16, according to the user's choice.

Preferentially, the seat 3A is produced by restricting the cross-section of the orifice forming the housing 3, said restriction of the cross-section forming a bearing surface against which the head 15A, 16A of the screw, wherein the diameter is greater than that of the shaft 15B, 16B extending same, rests (see FIGS. 2 to 4). Preferentially, the seat 3A is produced at the bottom of the orifice forming the housing 3, i.e. at the bottom face 2B, such that the housing 3 is thus able to substantially contain the whole, or at the very least a majority fraction, of the head 15A, 16A received therein. This enables a thin profile assembly, which is for this reason non-traumatic.

According to an important characteristic of the invention, the seat 3A and the head 15A, 16A of each fixation member 15, 16 are all shaped with respect to each other so that:

on one hand, the cooperation of the seat 3A and the head 15A of the first fixation member 15 substantially locks the latter in a predetermined orientation with respect to the assembly part 2,

and, on the other, the cooperation of the seat 3A and the head 16A of the second fixation member 16 enables a variation of the orientation thereof with respect to the assembly part 2.

In other words, the kit 1 is designed to enable the use of two alternative configurations (according to the user's choice) on the first housing 3, each of these configurations making it possible to obtain a specific function.

In this way, in the first configuration (illustrated in FIG. 3), the first housing 3 cooperates with the first fixation member 15, i.e. the first housing 3 individually receives the head 15A of the first fixation member 15, such that the latter rests against the seat 3A. According to the invention, the shape of the seat 3A and the shape of the head 15A are designed with respect to each other to guide and substantially hold the fixation member 15 in a predetermined fixed orientation with respect to the assembly part 2.

This means that when the first housing 3 cooperates with the first fixation member 15, i.e. when the head 15A of the first fixation member 15 rests against the seat 3A of the housing 3, the angular position of the first fixation member 15 is fixed in a stable and definitive manner and cannot substantially be modified by the user.

In the second configuration (illustrated in FIG. 4), the first housing 3 cooperates in this case with the second fixation member 16, i.e. the first housing 3 individually receives the head 16A of the second fixation member 16, such that the latter rests against the seat 3A. The shape of the seat 3A and the shape of the head 16A are designed with respect to each other to enable a variation of the angular orientation of the second fixation member 16 with respect to the assembly part 2. Advantageously, in this second configuration, the seat 3A guides the head 16A which allows same to move rotationally along the three spatial axes, i.e. the resting of the part 16A on the seat 3A produces a ball joint type mechanical connection between said head 16A and the assembly part 2. By means of this technical measure, the shaft 16B which extends from the head 16A may be moved manually by the user in a solid angle to reach an angular position which is acceptable for the practitioner.

In this way, the invention is based on the use of a single seat 3A against which either a head 15A wherein the shape makes it possible to substantially lock the corresponding fixation member 15 in a predetermined orientation, or a head 16A allowing the corresponding fixation member 16 a certain angular freedom may rest.

In other words, the general principle of the invention is based on the possibility for the practitioner to choose, for a single housing 3 provided in the assembly part 2:

either a fixation member 15 favoring firm and stable attachment of the head 15A of the fixation member 15 and the assembly part 2, with however no possibility to choose an angular orientation of the fixation member with respect to the assembly part 2,

or an assembly wherein the mechanical strength is in principle lower but which allows the possibility to choose, in a given solid angle, the orientation of the fixation member 16 with respect to the assembly part 2.

Due to these characteristics, the practitioner is able, according to the anatomical situation, to choose the most suitable fixation member according to each individual case, favoring either stability at the plate/screw interface, or the use of precision screw positioning with respect to the plate and, above all, the bone, wherein the screw enters.

The invention is particularly advantageous if the assembly part 2 comprises a plurality of housings 4, 5, 6, 7, 8, 9, 10, 11, 12, 13, 14 similar to the first housing 3, each housing 4, 5, 6, 7, 8, 9, 10, 11, 12, 13, 14 being associated with two fixation members respectively similar to the first and second fixation members 15, 16. In this way, the practitioner is able to choose for each individual housing, according to the anatomical situation, the most suitable fixation member, making it possible to definitively obtain an excellent overall compromise between rigidity and stability of the fixation, on one hand, and versatility and adaptability to the anatomical situation, on the other.

In this way, in a particularly advantageous embodiment of the invention, the kit 1 comprises at least:

not only a first through housing 3 and associated first and second fixation members 15, 16, as described above,

but also, on one hand, at least one second through housing 4, of a substantially similar design to that of the first housing 3, said second housing 4 being preferentially identical to the first housing 3, and, on the other, at least one third fixation member of a substantially similar design to that of the first fixation member 15, and preferentially identical to that of said first fixation member 15, and a fourth fixation member of substantially similar design to that of the second fixation member 16, said third and fourth fixation members being designed to cooperate with the second housing 4 in the same way as said first and second fixation members 15, 16 cooperate with the first housing 3.

Naturally, there are a multitude of possibilities accessible to those skilled in the art to produce the dual mechanical function according to the invention.

According to the example illustrated in the figures, the seat 3A thus comprises a bearing surface of substantially tapered shape against which the head 15A, 16A of the selected fixation member 15, 16 is intended to rest. As illustrated in the figures, the tapered bearing surface forming the seat 3A has an axis of symmetry Y-Y′, which is preferentially perpendicular to the main extension plane of the assembly part 2. Naturally, it can be envisaged to provide a housing 3 extending transversely, with in this case an axis of symmetry Y-Y′ extending along a transverse incidence with respect to the main extension plane of the assembly part 2.

Advantageously, the head 15A of the first fixation member 15 comprises a bottom face 150A intended to rest against the seat 3A, said bottom face having a substantially tapered shape, and preferentially a tapered ring shape as illustrated in FIG. 5. Preferentially, the tapered shape of the bottom face 150A of the head 15A is complementary to the tapered shape of the seat 3A, i.e. the tapered shape of the bearing surface forming the seat 3A. This makes it possible, as illustrated in FIGS. 2 and 3, to produce a plane bearing surface of the bottom face 150A against the seat 3A. This plane bearing surface along a tapered interface makes it possible to lock the screw in a given orientation corresponding to the axis of symmetry Y-Y′ of the seat 3A, such that the extension axis X-X′ of the shaft 15B and the axis Y-Y′ are merged. In this way, this truncated/truncate contact enables particularly stable and durable attachment of the fixation member 15 with the assembly part 2.

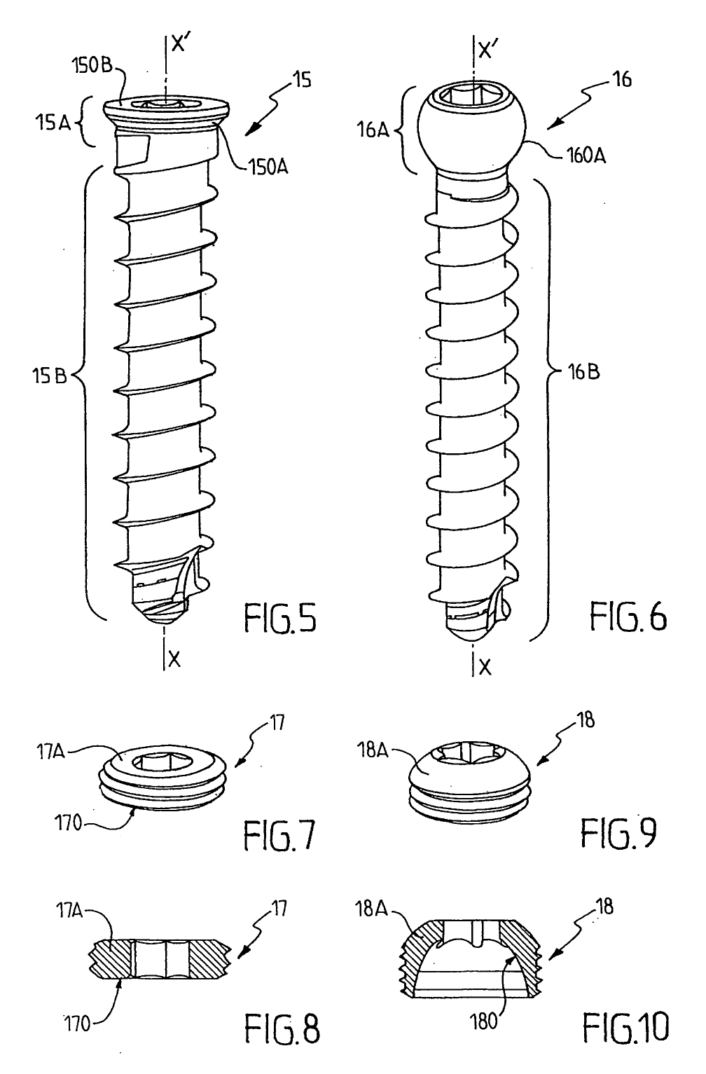

Advantageously, the head 16A of the second fixation member 16 also comprises a bottom face 160A intended to rest against the seat 3A, said bottom face 160A being in sphere portion form, as illustrated in FIG. 6. In this way, the spherical head 16A of the second fixation member 16 rests against the tapered bearing surface of the seat 3A along a substantially linear bearing surface area (see FIG. 4), such a linear bearing surface allowing an angular clearance of the second fixation member 16 with respect to the assembly part 2, i.e. a variation, in a solid angle, of the angle α between the extension axis X-X′ of the shaft 16B and the axis Y-Y′ attached to the seat 3A. In other words, the head 16A is housed in an orientable manner in the housing 3.

Advantageously, the kit 1 according to the invention also comprises at least one locking member 17, 18 designed to be fixed in the first housing 3 so as to rest against the head 15A, 16A of the selected fixation member 15, 16. In other words, the first housing 3 is advantageously designed to cooperate individually with the locking member 17, 18 such that said member applies a locking force on the head 15A, 16A of the selected fixation member, said force thus making it possible to carry out mechanical embedding of the head 15A, 16A with respect to the assembly part 2.

To this end, the locking member 17, 18 is fixed in the first housing 3 so that the head 15A, 16A of the selected fixation member 15, 16 is inserted between the seat 3A, on one hand, and the locking member 17, 18, on the other, said head 15A, 16A thus being compressed between said seat 3A and locking member 17, 18.

The use of a locking member 17, 18 makes it possible to seal the head 15A, 16A of the fixation member 15, 16 in question, this sealing helping to obtain an excellent mechanical stability of the assembly produced in this way. Such a stability is naturally very beneficial from a therapeutic point of view, as it particularly prevents mobility of the fixation member 15, 16 with respect to the assembly part 2, which could result in the extraction of the fixation member 15, 16 under the effect of the patient's movement.

The use of the locking member 17, 18 thus makes it possible, in combination with the other characteristics described above, to obtain a kit which, while being particularly simple, economical and versatile, displays an excellent mechanical stability irrespective of the manner in which it is used.

Advantageously, the first housing 3 comprises a threaded portion 3B, the locking member 17, 18 comprising a threaded pin 17A, 18A intended to be screwed in the threaded portion 3B.

More specifically, the housing 3A advantageously comprises a cylindrical orifice with an axis of symmetry X-X′ which extends from the top face 2A of the assembly part 2 to the seat 3A, which is located in the vicinity of the bottom face 2B. The inner wall of said cylindrical orifice is threaded, i.e. it comprises a hollow threading intended to cooperate with an outer threading provided in the threaded pin 17A, 18A on the lateral wall thereof, said pin also having a revolution symmetry as illustrated in the figures. When the pin 17A, 18A is screwed into the housing 3A, its axis of symmetry merges with the axis of symmetry X-X′ of the housing 3A.

It can be envisaged to use a single threaded pin wherein the design enables it to cooperate either with the first fixation member 15, if the user chooses the first configuration, or with the second fixation member 16, if the user chooses the second configuration.

However, in order to optimize the desired mechanical locking effect, it is preferable for the kit 1 according to the invention to comprise a first and a second locking member 17, 18 each comprising a locking face 170, 180 intended to rest against the head 15A, 16A when the locking member 17, 18 is fixed (in this case by screwing) in the housing 3, the locking face 170 of the first locking member 17 having a substantially complementary shape to that of the head 15A of the first fixation member 15, while the locking face 180 of the second locking member 18 has a substantially complementary shape to that of the head 16A of the second fixation member 16. In this way, in the example of an embodiment illustrated in the figures, the head 15A of the first fixation member 15 comprises a substantially flat top face, perpendicular to the extension axis X-X′ of the screw. The first locking member 17 intended to cooperate specifically with the first fixation member 15 then comprises a locking face 170, corresponding to the bottom face thereof, which is also substantially flat in shape, so as to favor a plane contact between the top face 150B of the head 15A and the locking face 170 of the threaded pin 17A forming the first locking member 17.

In the example illustrated in the figures, the head 16A of the second fixation member 16 has a substantially spherical shape, and more specifically the shape of a sphere truncated at the top part thereof. In this case, the locking face 180 of the second locking member 18 intended to cooperate specifically with the second fixation member 16 has a curved spherical shape, i.e. it corresponds to spherical contoured trough provided inside the second locking member 18, as illustrated in FIG. 10. This specific shape of the locking face 180 enables perfect congruence, and therefore surface support, of the locking face 180 on the screw head 16A. Such surface support is naturally liable to favor an excellent mechanical strength and therefore an optimized locking effect.

Finally, it is particularly advantageous to provide, for each housing 3, 4, 5, 6, 7, 8, 9, 10, 11, 12, 13, 14, two fixation members substantially identical to the first and second fixation members 15, 16 described above respectively and two locking members substantially identical to the first and second locking member 17, 18 described above respectively.

This enables the practitioner to choose, for each housing, the most suitable fixation member and the corresponding locking member.

What is claimed is:

1. Fixation kit (1) for medical or surgical use comprising an assembly part (2) intended to be coupled with a biological substrate and comprising at least one first through housing (3) provided therein, said kit (1) comprising a first and a second fixation member (15, 16) each comprising a head (15A, 16A) and a shaft (15B, 16B) intended to be anchored in said substrate, said kit (1) being characterized in that said first housing (3) is designed to cooperate equally well with either of said first and second fixation members (15, 16) according to the user's choice, said housing (3) being shaped to create a seat (3A) comprising a bearing surface of substantially tapered shape against which the head (15A, 16A) of the selected fixation member (15, 16) is intended to rest, the seat (3A) and the head (15A, 16A) of each fixation member (15, 16) being shaped with respect to each other so that, on one hand, the cooperation of the seat (3A) and the head (15A) of the first fixation member (15) substantially locks the latter in a predetermined orientation with respect to the assembly part (2), the head (15A) of the first fixation member (15) comprising a bottom face (150A) of substantially tapered shape intended to rest against the bearing surface of substantially tapered shape, and so that, on the other, the cooperation of the seat (3A) and the head (16A) of the second fixation member (16) enables a variation of the orientation thereof with respect to the assembly part (2).

2. Kit according to claim 1 characterized in that it also comprises at least one locking member (17, 18) designed to be fixed in the first housing (3) so as to rest against the head (15A, 16A) of the selected fixation member (15, 16).

3. Kit (1) according to claim 2 characterized in that the locking member (17, 18) is fixed in the first housing (3) so that the head (15A, 16A) of the selected fixation member (15, 16) is inserted between the seat (3A) and the locking member (17, 18).

4. Kit (1) according to claim 2 or 3 characterized in that the first housing (3) comprises a threaded portion (3B), the locking member (17, 18) comprising a threaded pin (17A, 18A) intended to be screwed in the threaded portion (3B).

5. Kit (1) according to any of claims 2 to 4 characterized in that it comprises a first and a second locking member (17, 18) each comprising a locking face (170, 180) intended to rest against the head (15A, 16A) when the locking member (17, 18) is fixed in the housing (3), the locking face (170) of the first locking member (17) having a substantially complementary shape to that of the head (15A) of the first fixation member (15), while the locking face (180) of the second locking member (18) has a substantially complementary shape to that of the head (16A) of the second fixation member (16).

6. Kit (1) according to any of claims 1 to 5 characterized in that the head (16A) of the second fixation member (16) also comprises a bottom face (160A) intended to rest against the seat (3A), said bottom face (160A) being in sphere portion form.

7. Kit (1) according to any of claims 1 to 6 characterized in that it comprises, on one hand, at least one second through housing (4), of a substantially similar design to that of the first housing (3), and, on the other, at least one third fixation member of a substantially similar design to that of the first fixation member (15), and a fourth fixation member of substantially similar design to that of the second fixation member (16), said third and fourth fixation members being designed to cooperate with the second housing (4) in the same way as said first and second fixation members (15, 16) cooperate with the first housing (3).

8. Kit (1) according to any of claims 1 to 7 characterized in that said first and second fixation members (15, 16) consist of a first and second screw, respectively.

9. Kit (1) according to any of claims 1 to 8 characterized in that said biological substrate consists of bone, while the assembly part (2) is an implant or surgical ancillary material.

10. Kit (1) according to claim 9 characterized in that the assembly part (2) is an osteosynthesis or arthrodesis plate.
